(19) United States
(12) Patent Application Publication
(10) Pub. No.: US 2006/0160767 A1
(43) Pub. Date: Jul. 20, 2006

(54) Method of providing food product with prolonged energy release

(75) Inventor: Marie-Helene Saniez nee Degrave, Lille (FR)

Correspondence Address: YOUNG & THOMPSON, 745 SOUTH 23RD STREET, 2ND FLOOR, ARLINGTON, VA, 22202, US

(73) Assignee: ROQUETTE FRERES, LESTREM (FR)

(21) Appl. No.: 11/218,625

(22) Filed: Sep. 6, 2005

Related U.S. Application Data
(62) Division of application No. 10/113,463, filed on Mar. 29, 2002.

(30) Foreign Application Priority Data
Mar. 30, 2001 (FR) 01 04412

(51) Int. Cl. A61K 31/716

(52) U.S. Cl. 514/58

(57) ABSTRACT

The subject of the present invention is a nutritive composition for prolonged release of energy, characterized in that it comprises between 50 and 99.9%, preferably 60 to 99.9% and still more preferably between 70 and 99.9% by weight of branched maltodextrins having between 15 and 35% of 1-6 glucoside linkages, a reducing sugar content of less than 20%, a polymolecularity index of less than 5 and a number-average molecular mass Mn at most equal to 4500 g/mol. The subject of the invention is also a food product containing the said nutritive composition.

This application is a division of co-pending application Ser. No. 10/113,463, filed on Mar. 29, 2002, the entire contents of which are hereby incorporated by reference.

### FIELD OF THE INVENTION

The subject of the present invention is a nutritive composition intended, in particular, for sportspeople, which is capable of constituting a prolonged energy supply. More precisely, the subject of the invention is a nutritive composition with prolonged energy release, containing a particular saccharide. The subject of the invention is also a food product containing said nutritive composition.

### BACKGROUND OF THE INVENTION

The capacity for exercise of long duration is limited by the glycogen content in the muscle. A linear relationship indeed exists between this content in the lower limbs and the period before exhaustion during exercise lasting from a few minutes to a few hours. Accordingly, among the objectives of training and nutritional preparation of sportspeople are the increase, before the activity, in the glycogen reserves in the muscles solicited by the exercise and the saving of muscle glycogen during the activity. It has been demonstrated that the carbohydrate diet before, during and after exercise was crucial in managing the muscle glycogen reserves. The nutritional strategies during the preparation of, during the course of and during recovery from intense and sustained activities take into account the kinetics and factors affecting the synthesis of muscle glycogen. These strategies are numerous and continue to evolve. They comprise, in general, three aspects: the preparation a few days before the activity whose objective is the optimum load of muscle and hepatic glycogen, the preparation within the hour preceding the exercise whose objective is to continue the constitution of hepatic glycogen reserves, and the control of muscle glycogenolysis during the activity. The supplies are different according to the aspect considered. As regards the supplies immediately before the effort and during the effort, to which the present invention relates, it is generally recommended to consume carbohydrates which solicit a minimum secretion of insulin so as not to disrupt the hormonal modifications necessary for the metabolic energy adaptations at the start of exercise. In the event of excessive supply of glucose, for example, the subject risks a hyper- and then a reactive hypoglycaemia within the first half hour of exercise through hyperinsulinaemia, which results in raging hunger, fatigue which may extend as far as discomfort and a need to reduce the pace, or even to stop. This same phenomenon was observed with carbohydrates such as maltodextrins. A solution of fructose is currently considered as the optimum drink just before the exercise, in solution up to 35 g/l. Fructose is less insulin-secretory and its intestinal absorption is slower.

### SUMMARY OF THE INVENTION

From this acknowledgment of the state of the art and seeking to develop an energizing composition intended for supplying just before and during the exercise, the Applicant has found, after numerous research studies, that it was possible to achieve this objective as long as the energizing composition comprised a particular saccharide capable, against all expectations, of constituting a sustained energy supply, that is to say from its consumption and throughout the exercise, without exhibiting the disadvantage of generating a rapid increase in glycaemia followed by a sharp hypoglycaemia.

### BRIEF DESCRIPTION OF THE DRAWING

FIG. 1 shows the progression of insulinaemia and glycaemia with the consumption of glucose and branched maltodextrins.

### DETAILED DESCRIPTION OF THE INVENTION

The subject of the invention is therefore a nutritive composition with prolonged energy release, characterized in that it comprises branched maltodextrins having between 15 and 35% of 1-6 glucoside linkages, a reducing sugar content of less than 20%, a polymolecularity index (ratio of weight-average molecular mass to number-average molecular mass) of less than 5, and a number-average molecular mass Mn at most equal to 4500 g/mol.

The expression branched maltodextrins is understood to mean for the purposes of the present invention the maltodextrins described in patent application EP 1,006,128 and in its US counterpart (U.S. Ser. No. 09/455,009) of which the Assignee is the proprietor. The entire content of U.S. Ser. No. 09/455,009 is herein incorporated by reference. These branched maltodextrins have an indigestibility character which has the consequence of reducing their calorific value, by preventing their assimilation in the small intestine. Their low content of molecules having a low degree of polymerization (“DP”) also contributes to their low calorific value. Their content of 1-6 glucoside linkages is between 22 and 35% and has the consequence of reducing their cariogenic power by reducing their assimilation by the microorganisms of the buccal cavity. This high level of 1-6 linkages also confers quite particular prebiotic properties on them: it has indeed appeared that the bacteria of the caecum and of the colon of humans and animals, such as the butyrogenic, lactic or propionic bacteria, metabolize highly branched compounds. Moreover, these branched maltodextrins promote the development of bifidogenic bacteria to the detriment of the undesirable bacteria. This results in properties that are quite beneficial to the health of the consumer. These branched maltodextrins have, in addition, a relatively high content of residual 1-4 glucoside linkages. This content may be between 42 and 50%, which content, to the knowledge of the Applicant, has never been described in combination with a content of 1-6 linkages of between 22 and 35%. The branched maltodextrins thus have a ratio of 1-6/1-4 linkages of about 0.6.

The polymolecularity index is given by the ratio of the weight-average molecular masses to the number-average molecular masses, the latter being measured, for example, by gel permeation chromatography.

The said branched maltodextrins have a polymolecularity index of less than 5, for a number-average molecular mass (Mn) of 500 to 4500 g/mol.

Their weight-average molecular mass (MW) is thus between 2000 and 6000 g/mol approximately.

Dextrins of high molecular weight (720,000 g/mol) and having a level of 1-6/1-4 linkages of 0.02 to 0.08 have been proposed in the document WO 95/22562 for the preparation of sportspeople before and after exercise. These dextrins are in fact intended to strengthen the storage of glycogen a few days before the exercise in the same manner as foods containing complex carbohydrates (pasta, rice).

The branched maltodextrins according to the invention being weakly digested in the small intestine, they supply energy slowly in the form of glucose to the individual consuming them. This glucose, the constituent unit of branched maltodextrins, then appears slowly in the bloodstream, and does not cause hypoglycaemia after ingestion. The supply of energy continues in the large intestine where the branched maltodextrins are fermented to short-chain fatty acids (termed volatile fatty acids) which participate in the energy metabolism. That is not the case for glucose or other simple carbohydrates which, for their part, are completely absorbed in the small intestine.

According to a preferred variant of the invention, the said branched maltodextrins have a reducing sugar content of between 2 and 5%, and an Mn of between 2000 and 3000 g/mol.

According to another preferred variant of the invention, all or some of the said branched maltodextrins are hydrogenated.

The nutritive composition according to the invention is primarily intended for sportspeople taking part in endurance exercises, but it is also quite suitable for the prolonged energy supply of any individual, including children, requiring this energy source on different occasions.

The nutritive composition according to the invention may be ingested in various ways. It can indeed be provided in powdered form, packaged in sachets and optionally supplemented with additives such as, for example, mineral salts, vitamins, flavourings, colourings, so as to be ingested as it is or dissolved in a drink immediately before use. It may also be provided in the form of a ready-to-use syrup.

According to a preferred embodiment of the invention, the content of branched maltodextrins of the said nutritive composition is between 50 and 99.9% by weight, preferably between 60 and 99.9%, and still more preferably between 70 and 99.9% by weight.

The nutritive composition according to the invention may advantageously comprise a mixture of the said branched maltodextrins with any type of hypoglycaemic and/or hypoinsulinaemic saccharide such as, for example, fructose or tagatose. According to an advantageous variant, the said nutritive composition comprises a mixture of the said branched maltodextrins with one or more pentoses, it being possible for this mixture to be formulated in powder form or in liquid form, knowing that, in this case, the bacteriological stability of the branched maltodextrins in liquid form is thereby advantageously markedly improved. It is possible, for example, to prepare mixtures of branched maltodextrins according to the invention with, for example, fructose syrups such as the MELIOSE® 700 marketed by the Assignee. The proportions of the mixture vary according to the final formulation for which the said mixture is intended. By way of example, mixtures may be prepared comprising up to 50% of fructose, in the form of syrups of about 70% dry matter.

The nutritive composition according to the invention may also be incorporated into a food product consisting in particular of an energizing bar, a cereal bar, a biscuit, a drink, a dietetic supplement, a breakfast cereal composition, a tablet, a confectionery, a yoghurt or a dessert cream. Preferably, the said food product is a bar, a biscuit, a drink or a confectionery. The food product according to the invention advantageously comprises 0.5 to 75%, and preferably 0.5 to 30% of the said nutritive composition.

The subject of the invention is also the use of a nutritive composition according to any one of claims 1 to 3 to supply the prolonged energy release. When it is ingested by an individual (sportsperson or child for example), the nutritive composition according to the invention provides energy resulting in an increase in post-prandial glycaemia without causing a period of hypoglycaemia, this energy being released in the body in a prolonged manner, that is to say at the moment of its consumption and long afterwards.

The invention will be understood more clearly on reading the examples which follow and the figure relating thereto, which are given by way of illustration and without limitation.

### EXAMPLE 1

### Study of Glycaemia and Insulinaemia

6 healthy individuals received either 50 g of glucose or 50 g of a nutritive composition containing 95% by dry weight of branched maltodextrins whose composition is the following:

| | |
|---|---|
| reducing sugars | 2.3 |
| Mn (g/mol) | 2480 |
| Mw (g/mol) | 5160 |
| 1,2 linkage (%) | 10 |
| 1,3 linkage (%) | 12 |
| 1,4 linkage (%) | 49 |
| 1,6 linkage (%) | 29 |

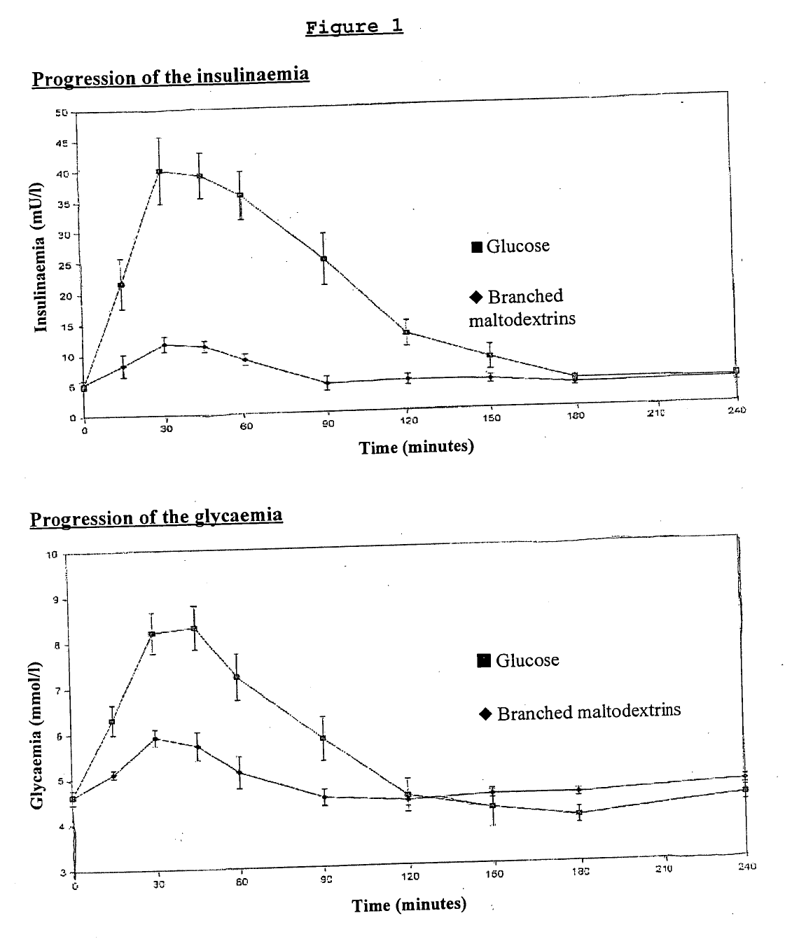

The glycaemia and the insulinaemia are measured before consumption of the carbohydrate, and then at the times 15,30,45,60,90,120,150,180 and 240 minutes after ingestion. The curves of glycaemia and insulinaemia as a function of time are established for each carbohydrate and are represented in FIG. 1.
The maximum glycaemia and insulinaemia are obtained 30 minutes after ingestion of the carbohydrate, regardless of its nature.
The glycaemia and insulinaemia return to the base line within 90 minutes in the individuals who received the nutritive composition according to the invention, against 120 minutes for the individuals who received glucose.
The glycaemia remains constant from time 90 to time 240 in the individuals who received the nutritive composition according to the invention, whereas it decreases further in the case of glucose and rises only at the time 240 without having reached the base line. The insulinaemia decreases further in the case of glucose, whereas it remains constant, at the base line in the case of the branched maltodextrins.

These results demonstrate that the ingestion of a nutritive composition in accordance with the invention causes an increase in post-prandial glycaemia which can therefore supply energy without causing hypoglycaemia. The insulinaemia is, for its part, markedly less compared with glucose.

### EXAMPLE 2

### Preparation of Food Products Comprising the Nutritive Composition in Accordance with Example 1

1) Cereal Bar

Binding caramel recipe

| | | Composition of |
|---|---|---|
| | Ingredient used | the finished |
| | (by weight) | product (%) |
| Powdered milk | 101 | 10 |
| Sucrose | 300 | 32 |
| Nutritive composition | 126 | 12.7 |
| NaCl | 4 | 0.4 |
| Water | 40 | |
| NEOSORB 70/70* | 118 | 8.8 |
| FLOLYS E7081S* | 132 | 11 |
| M.G.V.** COSE | 174 | 18 |
| Soyabean lecithin | 5 | 0.5 |
| Residual moisture | | 6.6 |
| Total | 1000 | 100 |

*marketed by the Applicant
**vegetable fats

Procedure
Mix the whole milk powder with the sucrose (M1)
Mix the water, the NEOSORB 70/70 and the FLOLYS E7081S (M2)
Mix the vegetable fat (previously melted at 50° C.), the lecithin and the branched maltodextrins (M3)
With stirring, add the mixture M1 to the mixture M2 and then cook on a low fire until 110° C. is reached
When the temperature of 110° C. has been reached, gradually pour in the mixture M3
A drop in temperature (about 5° C.) and an increase in the viscosity of the final mixture are then observed.
Still on a low fire and with vigorous stirring, cook the final mixture until 110° C. is reached.
Mix the caramel with the cereals, observing the prescribed proportions (75/25).
Pour on a slab
Cool, cut, wrap.

2) Drink

A sparkling drink is prepared which contains the nutritive composition in accordance with the invention comprising 95% by weight of branched maltodextrins.

Quantities in grams per 1 litre of drink:

| | Drink according to |
|---|---|
| | the invention |
| nutritive composition | 73.68 |
| aspartame | 0.118 |
| acesulfame K | 0.118 |
| lemon flavour | 0.7 |
| citric acid (dry) | 1.9 |
| sodium benzoate (dry) | 0.09 |
| water at 4° C. | qs 11 |

Aspartame: NUTRASWEET
Acesulfame K: HOECHST
Lemon flavour: AG 31711 (QUEST)

0.5 litre of aerated water is prepared. The sweeteners and the nutritive composition according to the invention are then added. The rest of the ingredients are then incorporated and water is added to a volume of 1 litre.

3) Biscuit

Biscuits are prepared which contain the nutritive composition in accordance with the invention, comprising 95% by dry weight of branched maltodextrins having a similar composition to that of Example 1.

Quantities per 100 g of dough:

| | Biscuit according to |
|---|---|
| Ingredient | the invention |
| flour | 55.8 |
| fat | 12.4 |
| sucrose | 15.1 |
| nutritive composition | 6.2 |
| water | 9.3 |
| ammonium bicarbonate | 0.3 |
| sodium bicarbonate | 0.2 |
| sodium pyrophosphate | 0.2 |
| salt | 0.3 |
| vanilla | 0.1 |
| lecithin | 0.1 |

Procedure:
Weigh the water, the ammonium bicarbonate and the sodium bicarbonate. Mix for 5 minutes in a Hobart kneader on speed 1.
Add the fat and the soya bean lecithin, and stir for one minute on speed 1, and then 4 minutes on speed 2.
Weigh the rest of the powders (flour, sucrose, nutritive composition, sodium hydrogen pyrophosphate, salt and vanilla flavour). Mix them, and then add them to the kneader. Stir for 10 minutes on speed 1, with one interruption to scrape the sides of the kneader and the stirring blade.
Form the biscuits in a rotary moulder and place them on a baking tray.
Heat in a rotary oven at 200° C. for 10 minutes.
Allow to cool at 25° C.

4) Dessert Cream

Ingredients Used:

| Ingredients | Percentage by weight used |
|---|---|
| powdered skimmed milk | 9 |
| nutritive composition | 5.7 |
| acesulfame K | 0.04 |
| starch * | 3 |
| carrageenan | 0.02 |
| vanilla flavour | 0.01 |
| water | 82.23 |
| total | 100 |

Starch: CLEARAM®CH3010 marketed by the Assignee.

25 litres of water are poured into the stirring tank of a tubular exchanger (VOMATEC B.V.) and then the milk powder is added. It is allowed to become hydrated for 20 minutes.

The tank is heated to 40° C. and the rest of the ingredients are added.

After a period of stirring of about 20 minutes, the mixture is sterilized in the tubular exchanger where it undergoes a heat treatment of 130° C. for 1 minute 30 seconds.

The dessert cream is recovered at the outlet of the apparatus in sterile pots.

1. A method for supplying prolonged energy release without causing a period of hypoglycemia, said method comprising the ingestion by an individual of a nutritive composition comprising between 50 to 99.9% by weight of branched maltodextrins having between 15 and 35% of 1-6 glucoside linkages, a reducing sugar content of less than 20%, a polymolecularity index of less than 5 and a number-average molecular mass Mn at most equal to 4500 g/mol.

2. The method of claim 1 wherein the nutritive composition have a reducing sugar content of between 2 and 5% and a Mn of between 2000 and 3000 g/mol.

3. The method of claim 1 wherein the nutritive composition comprises a mixture of the said branched maltodextrins with at least one hypoinsulinaemic monosaccharide.

4. The method of claim 2 wherein the nutritive composition comprises a mixture of the said branched maltodextrins with at least one hypoinsulinaemic monosaccharide.
